(19) United States
(12) Patent Application Publication
(10) Pub. No.: US 2022/0181015 A1
(43) Pub. Date: Jun. 9, 2022

(54) MONITORING GLUCOSE LEVEL USING DIFFERENT GRAPHICAL REPRESENTATIONS

(71) Applicant: Medtronic MiniMed, Inc., Northridge, CA (US)

(72) Inventors: Natassia Mattoon, La Mirada, CA (US); David Legray, Northridge, CA (US); Joseph P. Williams, Northridge, CA (US); Yuxiang Zhong, Arcadia, CA (US)

(21) Appl. No.: 17/544,666

(22) Filed: Dec. 7, 2021

Related U.S. Application Data
(60) Provisional application No. 63/123,355, filed on Dec. 9, 2020.

(51) Int. Cl. G16H 40/63; A61B 5/00; A61B 5/145

(52) U.S. Cl. CPC G16H 40/63; A61B 5/14532; A61B 5/743

(57) ABSTRACT

A system for monitoring a patient includes a memory and processing circuitry coupled to the memory. The processing circuitry is configured to determine a glucose level of the patient and determine a first graphical representation of the glucose level for display on a patient device based on the glucose level of the patient. The processing circuitry is further configured to determine a second graphical representation of the glucose level for display on a wearable device based on a target range of glucose levels. The second graphical representation is different from the first graphical representation. The processing circuitry is further configured to output an instruction to cause the wearable device to display the second graphical representation.

This application claims the benefit of US Provisional Patent Application No. 63/123,355, filed 9 Dec. 2020, the entire contents of which is incorporated herein by reference.

### TECHNICAL FIELD

The disclosure relates to medical systems and, more particularly, to medical systems for monitoring physiological parameters of a patient for the treatment, management, and/or prevention of disease, including but not limited to, diabetes, heart failure, and/or neural disorders.

### BACKGROUND

A patient with diabetes receives insulin from a pump or injection device to control the glucose level in his or her bloodstream. Naturally produced insulin may not control the glucose level in the bloodstream of a diabetes patient due to insufficient production of insulin and/or due to insulin resistance. To control the glucose level, a patient's therapy routine may include dosages of basal insulin and bolus insulin. Basal insulin, also called background insulin, tends to keep blood glucose levels at consistent levels during periods of fasting and is a long acting or intermediate acting insulin. Bolus insulin may be taken specifically at or near mealtimes or other times where there may be a relatively fast change in glucose level, and may therefore serve as a short acting or rapid acting form of insulin dosage.

### SUMMARY

Various aspects of techniques for assisting in management of glucose levels in a patient are described. With such techniques, a diabetes management system may display glucose levels, automatically generate insights related to glucose level trends, facilitate food logging, and automatically calculate recommended insulin doses. For example, one or more patient devices (e.g., smart phone, smart watch, laptop, tablet, computer) of the diabetes management system may display real-time (or near real-time) glucose levels received from one or more glucose sensors. The one or more patient devices can automatically log nutrition information (e.g., log macro nutrients such as carbohydrate amounts) for food intake events. In some examples, the system can automatically detect meal events based on detected eating gestures of the user and automatically generate insulin bolus reminders and/or recommendations based on the detected meal events. The system can also generate glucose pattern insights on how the user's activities positively or adversely impact glucose levels. The system can also output information about the time or percent of time the user's glucose are in range (e.g., between 180 mg/dL and 70 mg/dL), the time or percent of time the user's glucose are above a high threshold (e.g., 180 mg/dL), the time or percent of time the user's glucose are below a low threshold (e.g., 70 mg/dL), and/or the average glucose level.

Devices, systems, and techniques for managing glucose level and other physiological parameters in a patient are described. Medical devices (e.g., pump or injection device) may be in communication with, or otherwise use monitors having electrodes to perform various measurements for a patient. Such medical devices may include, for example, a percutaneous cannula configured to deliver insulin to the patient. The monitors (including but not limited to a continuous glucose monitor (CGM)) having electrodes may concurrently monitor the patient's response to treatment introduced by such medical devices. It may be desirable to quickly respond to a glucose level that is outside of a target range of glucose levels. For instance, if a glucose level that is greater than 200 mg/dL, a medical device may deliver insulin to the patient to reduce the glucose level. If a glucose level is less than 80 mg/dL, the medical device or monitor may recommend the patient consume food to increase the glucose level.

The techniques of this disclosure include a system configured to monitor a glucose level and to cause a patient device to display a first graphical representation of the glucose level and to cause a wearable device (e.g., a smart watch) to display a second graphical representation of the glucose level that is different from the first graphical representation. For example, the system may cause a watch face to display one or more arrows indicating a “spike” or drop in glucose level for a patient. In another example the system may cause the watch face to display a numeric value for the glucose level in a color that indicates that the glucose level is outside of the safe range (e.g., orange, red). In this way, a system may notify the patient of a glucose level that is projected to be outside of a target range in a convenient manner, which may improve a patient's satisfaction.

In one example, this disclosure describes a system for monitoring a patient, the system comprising a memory and processing circuitry coupled to the memory. The processing circuitry is configured to determine a glucose level of the patient and determine a first graphical representation of the glucose level for display on a patient device based on the glucose level of the patient. The processing circuitry is further configured to determine a second graphical representation of the glucose level for display on a wearable device based on a target range of glucose levels. The second graphical representation is different from the first graphical representation. The processing circuitry is further configured to output an instruction to cause the wearable device to display the second graphical representation.

In another example, this disclosure describes, a method for monitoring a patient includes determining, by processing circuitry, a glucose level of the patient and determining, by the processing circuitry, a first graphical representation of the glucose level for display on a patient device based on the glucose level of the patient. The method further includes determining, by the processing circuitry, a second graphical representation of the glucose level for display on a wearable device based on a target range of glucose levels. The second graphical representation is different from the first graphical representation. The method further includes outputting, by the processing circuitry, an instruction to cause the wearable device to display the second graphical representation.

In one example, this disclosure describes a patient device for monitoring a patient, the patient device comprising a user interface, a memory, and processing circuitry coupled to the memory and the user interface. The processing circuitry is configured to determine a glucose level of the patient and determine a first graphical representation of the glucose level for display on the user interface based on the glucose level of the patient. The processing circuitry is configured to determine a second graphical representation of the glucose level for display on a wearable device based on a target range of glucose levels. The second graphical representation is different from the first graphical representation. The processing circuitry is configured to output an instruction to cause the wearable device to display the second graphical representation.

The details of one or more aspects of the disclosure are set forth in the accompanying drawings and the description below. Other features, objects, and advantages of this disclosure will be apparent from the description and drawings, and from the claims.

### BRIEF DESCRIPTION OF DRAWINGS

FIG. 1 is a block diagram illustrating an example system for delivering or guiding therapy dosage, in accordance with one or more examples described in this disclosure.

FIG. 2 is a block diagram illustrating another example system for delivering or guiding therapy dosage, in accordance with one or more examples described in this disclosure.

FIG. 3 is a block diagram illustrating another example system for delivering or guiding therapy dosage, in accordance with one or more examples described in this disclosure.

FIG. 4 is a block diagram illustrating an example of a patient device, in accordance with one or more examples described in this disclosure.

FIG. 5 is a conceptual diagram illustrating a first example of a patient device and a wearable device displaying different graphical indications, in accordance with one or more examples described in this disclosure.

FIG. 6 is a conceptual diagram illustrating a second example of a patient device and a wearable device displaying different graphical indications, in accordance with one or more examples described in this disclosure.

FIG. 7 is a conceptual diagram illustrating a third example of a patient device and a wearable device displaying different graphical indications, in accordance with one or more examples described in this disclosure.

FIG. 8 is a conceptual diagram illustrating a fourth example of a patient device and a wearable device displaying different graphical indications, in accordance with one or more examples described in this disclosure.

FIG. 9 is a flow chart illustrating an example process, in accordance with one or more examples described in this disclosure.

FIG. 10 is a diagram illustrating an example system for monitoring and managing glucose levels of a user, in accordance with one or more examples described in this disclosure.

FIG. 11 is a diagram illustrating an example graphical user interface of a patient device for logging meal nutrition information, in accordance with one or more examples described in this disclosure.

FIG. 12 is a diagram illustrating example user interfaces of two patient devices, in accordance with one or more examples described in this disclosure.

FIG. 13 is a diagram illustrating an example graphical user interface of a patient device including an insulin dose recommendation, in accordance with one or more examples described in this disclosure.

FIG. 14 is a diagram illustrating example user interfaces of a patient device, in accordance with one or more examples described in this disclosure.

### DETAILED DESCRIPTION

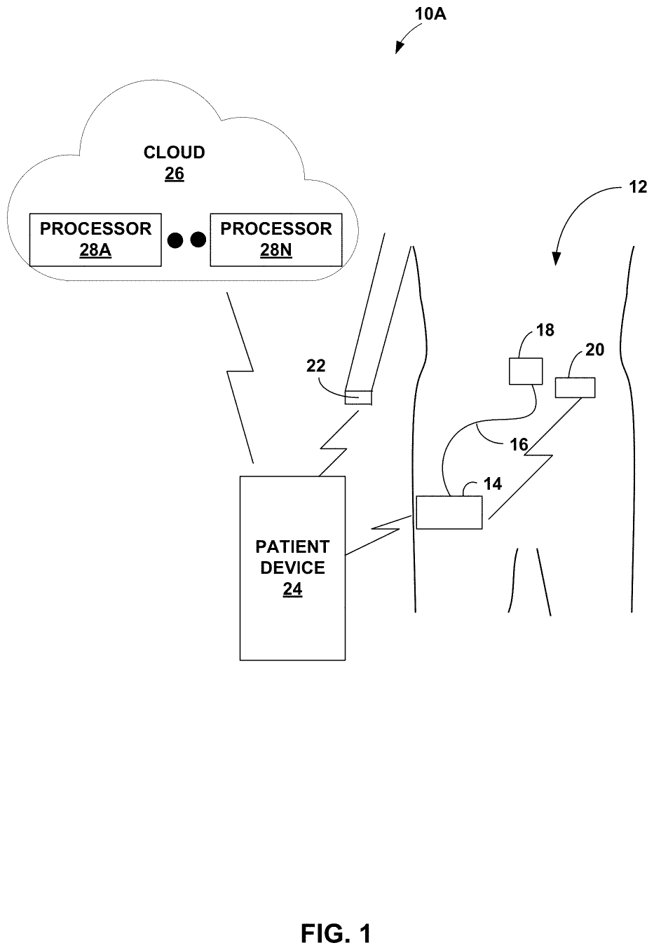

FIG. 1 is a block diagram illustrating an example system for delivering or guiding therapy dosage, in accordance with one or more examples described in this disclosure. FIG. 1 illustrates system 10A that includes patient 12, insulin pump 14, tubing 16, infusion set 18, sensor device 20, which may be a glucose sensor, wearable device 22, patient device 24, and cloud 26. Cloud 26 represents a local, wide area or global computing network including one or more processors 28A-28N (also, referred to herein as “processing circuitry 28”). In some examples, the various components may determine changes to therapy based on determination of glucose level by sensor device 20, and therefore system 10A may be configured to perform glucose sensing. In some examples, system 10A may be referred to as a continuous glucose monitoring (CGM) system 10A. Additionally, system 10A may be configured for co-morbidity management (e.g., management of a heart disease and/or management of a kidney disease) in accordance with one or more techniques described herein. As described herein, system 10A may, in some examples, provide CGM and/or treatment for co-morbidity management. However, in some examples, system 10A may support another device and/or a healthcare professional that provides CGM and/or treatment for co-morbidity management.

Patient 12 may be diabetic (e.g., Type 1 diabetic or Type 2 diabetic), and therefore, the glucose level in patient 12 may be uncontrolled without delivery of supplemental insulin. For example, patient 12 may not produce sufficient insulin to control the glucose level or the amount of insulin that patient 12 produces may not be sufficient due to insulin resistance that patient 12 may have developed.

To receive the supplemental insulin, patient 12 may carry insulin pump 14 that couples to tubing 16 for delivery of insulin into patient 12. Infusion set 18 may connect to the skin of patient 12 and include a cannula to deliver insulin into patient 12. Sensor device 20 may be a continuous glucose monitoring device (or continuous glucose monitor (CGM)) that, together with patient device 24 and/or processing circuitry 28, for a CGM system. One example of sensor device 20 is Guardian Sensor 3™ by Medtronic MiniMed, Inc. However, other examples of insulin pump systems may be used and the example techniques should not be considered limited to the Guardian™ Sensor 3. Sensor device 20 may be coupled to patient 12 to measure the glucose level in patient 12. For example, sensor device 20 may include one of more sensing components (e.g., electrodes) that can be percutaneously inserted into subcutaneous tissue to sense glucose levels and/or other physiological signals or conditions. Insulin pump 14, tubing 16, infusion set 18, and sensor device 20 may together form an insulin pump system. One example of the insulin pump system is the MINIMED™ 670G INSULIN PUMP SYSTEM by Medtronic MiniMed, Inc. However, other examples of insulin pump systems may be used and the example techniques should not be considered limited to the MINIMED™ 670G INSULIN PUMP SYSTEM.

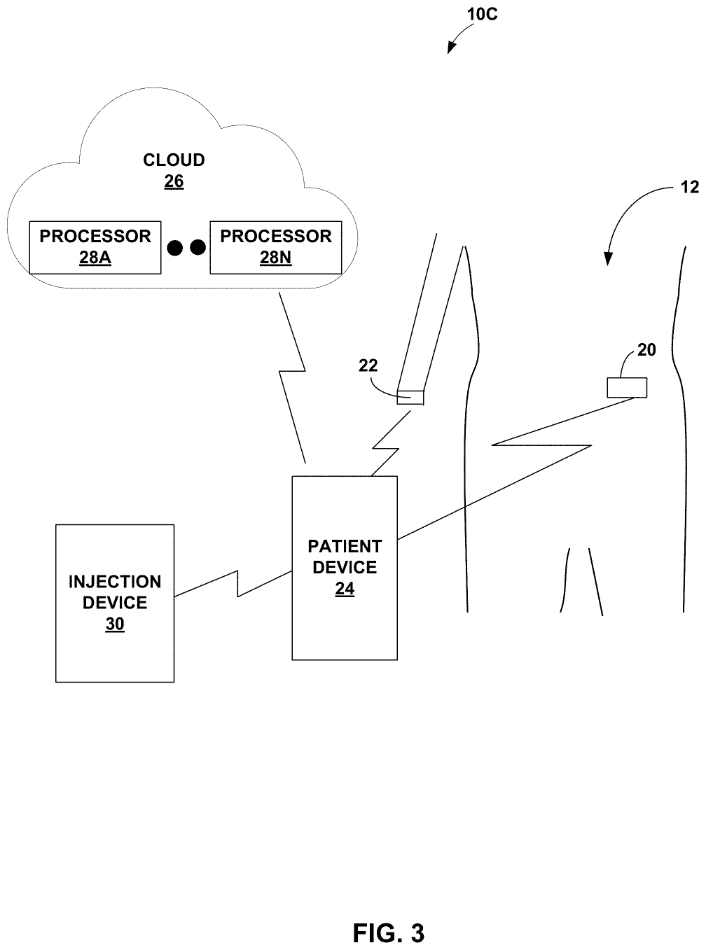

For example, the techniques described in this disclosure may be utilized in insulin pump systems that include wireless communication capabilities. Additionally, techniques described in this disclosure may also be utilized in other health monitoring and/or blood glucose management systems that may include, but are not limited to, CGMs in wired or wireless communication with insulin injection device 30 (FIG. 3) such as an insulin pen (including, but not limited to smart pens such as the InPen™ device), or CGMs in wireless communication with diabetes or health management applications configured to be capable of running on standalone health devices or consumer electronic devices (embodied, for example, as a patient device 24) including, but not limited to, a wearable device 22 such as a smartwatch, smartphone, or other personal computing device. However, the example techniques should not be considered limited to insulin pump systems or smart insulin pens with wireless communication capabilities, and other types of communication, such as wired communication, may be possible. In another example, insulin pump 14, tubing 16, infusion set 18, and/or sensor device 20 may be contained in the same housing, including, but not limited to single-use housings such as disposable “patch pumps” having an integrated pump and glucose monitoring system in a single form factor.

Insulin pump 14 may be a relatively small device that patient 12 can place in different locations. For instance, patient 12 may clip insulin pump 14 to the waistband of pants worn by patient 12. In some examples, to be discreet, patient 12 may place insulin pump 14 in a pocket. In general, insulin pump 14 can be worn in various places and patient 12 may place insulin pump 14 in a location based on the particular clothes patient 12 is wearing.

To provide insulin, insulin pump 14 includes one or more reservoirs (e.g., two reservoirs). A reservoir may be a plastic cartridge that holds up to N units of insulin (e.g., up to 300 units of insulin) and is locked into insulin pump 14. Insulin pump 14 may be a battery powered device that is powered by replaceable and/or rechargeable batteries.

Tubing 16, sometimes called a catheter, connects on a first end to a reservoir in insulin pump 14 and connects on a second end to infusion set 18. Tubing 16 may carry the insulin from the reservoir of insulin pump 14 to patient 12. Tubing 16 may be flexible, allowing for looping or bends to minimize concern of tubing 16 becoming detached from insulin pump 14 or infusion set 18 or concern from tubing 16 breaking.

Infusion set 18 may include a thin cannula that patient 12 inserts into a layer of fat under the skin (e.g., subcutaneous connection). Infusion set 18 may rest near the stomach of patient 12. The insulin travels from the reservoir of insulin pump 14, through tubing 16, and through the cannula in infusion set 18, and into patient 12. In some examples, patient 12 may utilize an infusion set insertion device. Patient 12 may place infusion set 18 into the infusion set insertion device, and with a push of a button on the infusion set insertion device, the infusion set insertion device may insert the cannula of infusion set 18 into the layer of fat of patient 12, and infusion set 18 may rest on top of the skin of the patient with the cannula inserted into the layer of fat of patient 12.

Sensor device 20 may include a cannula that is inserted under the skin of patient 12, such as near the stomach of patient 12 or in the arm of patient 12 (e.g., subcutaneous connection). Sensor device 20 may be configured to measure, using one or more electrodes inserted under the skin and/or into tissue of the patient, the interstitial glucose level, which is the glucose found in the fluid between the cells of patient 12. Sensor device 20 may be configured to continuously or periodically sample the glucose level and rate of change of the glucose level over time. In some examples, it may be possible for sensor device 20 to measure blood glucose level. The example techniques are described with respect to sensor device 20 measuring interstitial glucose level, but the techniques are not so limited.

In one or more examples, insulin pump 14 and sensor device 20, and the various components illustrated in FIG. 1, may together form a closed-loop therapy delivery system. For example, patient 12 may set a target glucose level, usually measured in units of milligrams per deciliter, on insulin pump 14. Insulin pump 14 may receive the current glucose level from sensor device 20, and in response may increase or decrease the amount of insulin delivered to patient 12. For example, if the current glucose level is higher than the target glucose level, insulin pump 14 may increase the insulin. If the current glucose level is lower than the target glucose level, insulin pump 14 may temporarily cease delivery of the insulin. Insulin pump 14 may be considered as an example of an automated insulin delivery (AID) device. Other examples of AID devices may be possible, and the techniques described in this disclosure may be applicable to other AID devices.

For example, insulin pump 14 and sensor device 20 may be configured to operate together to mimic some of the ways in which a healthy pancreas works. Insulin pump 14 may be configured to deliver basal insulin dosages, which is a small amount of insulin released continuously throughout the day. There may be times when glucose levels increase, such as due to eating or some other activity that patient 12 undertakes. Insulin pump 14 may be configured to deliver bolus insulin dosages on demand in association with food intake or to correct an undesirably high glucose level in the bloodstream. In one or more examples, if the sensed glucose level rises above a target level, then insulin pump 14 may increase the bolus insulin dosage to address the increase in glucose level. Insulin pump 14 may be configured to compute basal and bolus insulin delivery dosages, and deliver the basal and bolus insulin dosages accordingly. For instance, insulin pump 14 may determine the amount of basal insulin dosage to deliver continuously, and then determine the amount of bolus insulin dosage to deliver to reduce glucose level in response to an increase in glucose level due to eating or some other event.

Accordingly, in some examples, sensor device 20 may sample glucose level and rate of change in glucose level over time. Sensor device 20 may output the glucose level to insulin pump 14 (e.g., through a wireless link connection like IEEE 802.11, Wi-Fi™, Bluetooth™ or Bluetooth Low Energy (BLE)). Insulin pump 14 may compare the glucose level to a target glucose level (e.g., as set by patient 12 or clinician), and adjust the insulin dosage based on the comparison. In some examples, sensor device 20 may also output a projected glucose level (e.g., where glucose level is expected to be in the next 30 minutes (or any other period of time in the future)), and insulin pump 14 may adjust insulin delivery based on the predicted glucose level. Rather than or in addition to sensor device 20 outputting the projected glucose level, another device such as insulin pump 14, patient device 24, cloud 26, or processing circuitry 28 may determine and/or output the projected glucose level based on a current glucose level and one or more previous glucose levels from sensor device 20.

As described above, patient 12 or a clinician may set the target glucose level on insulin pump 14 or by communication with the patient via application running on the patient device 24. There may be various ways in which patient 12 or the clinician may set the target glucose level on insulin pump 14. As one example, patient 12 or the clinician may utilize patient device 24 to communicate with insulin pump 14. Examples of patient device 24 include, but are not limited to mobile devices, such as smartwatches, smartphones, tablet computers, laptop computers, and the like. Additionally, techniques described in this disclosure may also be utilized in other health monitoring and/or blood glucose management systems different from an insulin pump 14, including, but are not limited to, CGMs in wireless communication with insulin injection pens 30 (FIG. 3) (including, but not limited to smart pens such as the InPen™ device), or CGMs in wireless communication with diabetes or health management applications configured to be capable of running on standalone health devices or patient devices 24 including, but not limited to, smartwatches, smartphones, or other personal computing devices. In some examples, patient device 24 may be a special programmer or controller for insulin pump 14. Although FIG. 1 illustrates one patient device 24, in some examples, there may be a plurality of patient devices. For instance, system 10A may include a mobile device and a controller, each of which are examples of patient device 24. For ease of description only, the example techniques are described with respect to patient device 24, with the understanding that patient device 24 may be one or more patient devices.

Patient device 24 may be communicatively coupled with sensor device 20. For example, patient device 24 may be communicatively coupled with sensor device 20 via a wireless communication protocol (e.g., Wi-Fi™, IEEE 802.11, Bluetooth™, or BLE). As one example, patient device 24 may receive information from sensor device 20 through insulin pump 14, where insulin pump 14 relays the information between patient device 24 and sensor device 20. As another example, patient device 24 may receive information (e.g., glucose level or rate of change of glucose level or other physiological parameters derived from impedance measurement)directly from sensor device 20 (e.g., through a wireless link).

In one or more examples, patient device 24 may display a user interface with which patient 12 or the clinician may control insulin pump 14. For example, patient device 24 may be provided with an application or other graphical user interface for displaying a screen that allows patient 12 or the clinician to enter the target glucose level. As another example, patient device 24 may display a screen that outputs the current and/or past glucose level. In some examples, patient device 24 may output notifications to patient 12, such as notifications if the sensed glucose level is too high or too low, as well as notifications regarding any action that patient 12 needs to take. For example, if the batteries of insulin pump 14 or other medicament dispensing device, such as a smart insulin pen 30, are low on charge, then insulin pump 14 or pen 30 may output a low battery indication to patient device 24, and patient device 24 may in turn output a notification to patient 12 to replace or recharge the batteries.

Controlling insulin pump 14 through patient device 24 is one example, and should not be considered limiting. For example, insulin pump 14 may include a user interface (e.g., pushbuttons) that allows patient 12 or the clinician to set the various glucose levels of insulin pump 14. Also, in some examples, insulin pump 14 itself, or in addition to patient device 24, may be configured to output notifications to patient 12. For instance, if the sensed glucose level is too high or too low, insulin pump 14 may output an audible or haptic output. As another example, if the battery is low, then insulin pump 14 may output a low battery indication on a display of insulin pump 14.

The above describes examples ways in which insulin pump 14 may deliver insulin to patient 12 based on the current glucose levels (e.g., as measured by sensor device 20). In some cases, there may be therapeutic gains by proactively delivering insulin to patient 12, rather than reacting to when glucose levels become too high or too low.

The glucose level in patient 12 may increase due to particular user actions. As one example, the glucose level in patient 12 may increase due to patient 12 engaging in an activity like eating or exercising. In some examples, there may be therapeutic gains if it is possible to determine that patient 12 is engaging in the activity, and delivering insulin based on the determination that patient 12 is engaging in the activity.

For example, patient 12 may forget to cause insulin pump 14 to deliver insulin after eating, resulting an insulin shortfall. Alternatively, patient 12 may cause insulin pump 14 to deliver insulin after eating but may have forgotten that patient 12 previously caused insulin pump 14 to deliver insulin for the same meal event, resulting in an excessive insulin dosage. Also, in examples where sensor device 20 is utilized, insulin pump 14 may not take any action until after the glucose level is greater than a target level. By proactively determining that patient 12 is engaging in an activity, insulin pump 14 may be able to deliver insulin in such a manner that the glucose level does not rise above the target level or rises only slightly above the target level (i.e., rises by less than what the glucose level would have risen if insulin were not delivered proactively). In some cases, by proactively determining that patient 12 is engaging in an activity and delivering insulin accordingly, the glucose level of patient 12 may increase more slowly.

Although the above describes proactive determination of patient 12 eating and delivering insulin accordingly, the example techniques are not so limited. The example techniques may be utilized for proactively determining an activity that patient 12 is undertaking (e.g., eating, exercising, sleeping, driving, etc.). Insulin pump 14 may then deliver insulin based on the determination of the type of activity patient 12 is undertaking.

As illustrated in FIG. 1, patient 12 may wear wearable device 22. Examples of wearable device 22 include a smartwatch or a fitness tracker, either of which may, in some examples, be configured to be worn on a patient's wrist or arm. In one or more examples, wearable device 22 includes inertial measurement unit, such as a six-axis inertial measurement unit. The six-axis inertial measurement unit may couple a 3-axis accelerometer with a 3-axis gyroscope. Accelerometers measure linear acceleration, while gyroscopes measure rotational motion. Wearable device 22 may be configured to determine one or more movement characteristics of patient 12. Examples of the one or more movement characteristics include values relating to frequency, amplitude, trajectory, position, velocity, acceleration and/or pattern of movement instantaneously or over time. The frequency of movement of the patient's arm may refer to how many times patient 12 repeated a movement within a certain time (e.g., such as frequency of movement back and forth between two positions).

Patient 12 may wear wearable device 22 on his or her wrist. However, the example techniques are not so limited. Patient 12 may wear wearable device 22 on a finger, forearm, or bicep. In general, patient 12 may wear wearable device 22 anywhere that can be used to determine gestures indicative of eating, such as movement characteristics of the arm.

The manner in which patient 12 is moving his or her arm (i.e., the movement characteristics) may refer to the direction, angle, and orientation of the movement of the arm of patient 12, including values relating to frequency, amplitude, trajectory, position, velocity, acceleration and/or pattern of movement instantaneously or over time. As an example, if patient 12 is eating, then the arm of patient 12 will be oriented in a particular way (e.g., thumb is facing towards the body of patient 12), the angle of movement of the arm will be approximately a 90-degree movement (e.g., starting from plate to mouth), and the direction of movement of the arm will be a path that follows from plate to mouth. The forward/backward, up/down, pitch, roll, yaw measurements from wearable device 22 may be indicative of the manner in which patient 12 is moving his or her arm. Also, patient 12 may have a certain frequency at which patient 12 moves his or her arm or a pattern at which patient 12 moves his or her arm that is more indicative of eating, as compared to other activities, like smoking or vaping, where patient 12 may raise his or her arm to his or her mouth.

Although the above description describes wearable device 22 as being utilized to determine whether patient 12 is eating, wearable device 22 may be configured to detect movements of the arm of patient 12 (e.g., one or more movement characteristics), and the movement characteristics may be utilized to determine an activity undertaken by patient 12. For instance, the movement characteristics detected by wearable device 22 may indicate whether patient 12 is exercising, driving, sleeping, etc. As another example, wearable device 22 may indicate posture of patient 12, which may align with a posture for exercising, driving, sleeping, eating, etc. Another term for movement characteristics may be gesture movements. Accordingly, wearable device 22 may be configured to detect gesture movements (i.e., movement characteristics of the arm of patient 12) and/or posture, where the gesture and/or posture may be part of various activities (e.g., eating, exercising, driving, sleeping, etc.).

In some examples, wearable device 22 may be configured to determine, based on the detected gestures (e.g., movement characteristics of the arm of patient 12) and/or posture, the particular activity patient 12 is undertaking. For example, wearable device 22 may be configured to determine whether patient 12 is eating, exercising, driving, sleeping, etc. In some examples, wearable device 22 may output information indicative of the movement characteristics of the arm of patient 12 and/or posture of patient 12 to patient device 24, and patient device 24 may be configured to determine the activity patient 12 is undertaking.

Wearable device 22 and/or patient device 24 may be programmed with information that wearable device 22 and/or patient device 24 utilize to determine the particular activity patient 12 is undertaking. For example, patient 12 may undertake various activities throughout the day where the movement characteristics of the arm of patient 12 may be similar to the movement characteristics of the arm of patient 12 for a particular activity, but patient 12 is not undertaking that activity. As one example, patient 12 yawning and cupping his or her mouth may have a similar movement as patient 12 eating. Patient 12 picking up groceries may have similar movement as patient 12 exercising. Also, in some examples, patient 12 may be undertaking a particular activity, but wearable device 22 and/or patient device 24 may fail to determine that patient 12 is undertaking the particular activity.

Accordingly, in one or more examples, wearable device 22 and/or patient device 24 may “learn” to determine whether patient 12 is undertaking a particular activity. However, the computing resources of wearable device 22 and patient device 24 may be insufficient to performing the learning needed to determine whether patient 12 is undertaking a particular activity. It may be possible for the computing resources of wearable device 26 and patient device 24 to be sufficient to perform the learning, but for ease of description only, the following is described with respect to processing circuitry 28 in cloud 26.

As illustrated in FIG. 1, system 10A includes cloud 26 that includes processing circuitry 28. For example, cloud 26 includes a plurality of network devices (e.g., servers), and the plurality of devices each include one or more processors. Processing circuitry 28 may include one or more processors of the plurality of network devices, and may be located within a single one of the network devices, or may be distributed across two or more of the network devices. Cloud 26 represents a cloud infrastructure that supports processing circuitry 28 on which applications or operations requested by one or more users run. For example, cloud 26 provides cloud computing for using processing circuitry 28, to store, manage, and process data on the network devices, rather than by patient device 24 or wearable device 22. Processing circuitry 28 may share data or resources for performing computations, and may be part of computing servers, web servers, database servers, and the like. Processing circuitry 28 may be in network devices (e.g., servers) within a datacenter or may be distributed across multiple datacenters. In some cases, the datacenters may be in different geographical locations.

Processing circuitry 28 can include any one or more microprocessors, digital signal processors (DSPs), application specific integrated circuits (ASICs), field programmable gate arrays (FPGAs), or any other equivalent integrated or discrete logic circuitry, any other processing circuitry described herein, as well as any combinations of such components. The functions attributed processing circuitry 28, as well as other processing circuitry described herein, herein may be embodied as hardware, firmware, software or any combination thereof.

Processing circuitry 28 may be implemented as fixed-function circuits, programmable circuits, or a combination thereof. Fixed-function circuits refer to circuits that provide particular functionality, and are preset on the operations that can be performed. Programmable circuits refer to circuits that can be programmed to perform various tasks, and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits. Processing circuitry 28 may include arithmetic logic units (ALUs), elementary function units (EFUs), digital circuits, analog circuits, and/or programmable cores, formed from programmable circuits. In examples where the operations of processing circuitry 28 are performed using software executed by the programmable circuits, memory (e.g., on the servers) accessible by processing circuitry 28 may store the object code of the software that processing circuitry 28 receive and execute.

System 10A may be configured to determine a glucose level of patient 12. For example, sensor device 20 may determine a glucose level of patient 12 and output an indication of the glucose level to patient device 24. In this example, patient device 24 may receive the glucose level (e.g., a current glucose level or a projected glucose level) from sensor device 20, however, in other examples, patient device 24 may determine or estimate the glucose level, receive a patient reported glucose level, or receive an indication of the glucose from another device.

System 10A may determine a first graphical representation of the glucose level for display on patient device 24 based on the glucose level of patient 12. Patient device 24 may be configured to display the first graphical representation. For example, patient device 24 may display a graph of glucose levels for patient 12 over time. System 10A (e.g., one or more of processors 28, patient device, wearable device 22, or another device) may determine a second graphical representation of the glucose level for display on wearable device 22 based on a target range of glucose levels and output an instruction to cause wearable device 22 to display the second graphical representation. The second graphical representation may be different from the first graphical representation. System 10A may be configured to output the instructions to cause wearable device 22 to display the second graphical representation when patient device 24 displays the first graphical representation. That is, system 10A may be configured to simultaneously display the first graphical representation at patient device 24 and display the second graphical representation at wearable device 22.

System 10A may determine the first graphical representation to provide addition contextual information that is not displayed in the second graphical representation. For example, the graphical representation may include a graph of glucose levels for patient 12 over time. In some examples, the graph of glucose levels for patient 12 over time may be configured to display different time ranges based on a user input. In contrast, the second graphical representation may provide a current and/or projected glucose level without the additional context information (e.g., the graph). The second graphical representation may be more formed in a way to provide pertinent information in a way that is easier for the user to understand. In this way, a user (e.g., patient 12 or a caretaker of patient 12) may quickly identify pertinent information using the second graphical representation and determine further contextual information using the first graphical representation, which may improve a user satisfaction of system 10A. Moreover, wearable device 22 may comprise a smaller display than patient device 24. As such, system 10A may determine the second graphical display to display less information than the first graphical display to provide information in a format that is readable to a user. Similarly, system 10A may determine the first graphical representation to display more information than the second graphical representation to provide additional context and a richer display of information.

A display of the second graphical representation by wearable device 22 may generally be associated with the user taking immediate action (e.g., provides high level information that is easily consumable by the user). For example, patient device 24 may determine the second graphical representation to include a number of arrows or color to indicate whether a projected glucose level for the patient is outside of a target range. The first graphical representation may not include the number of arrows or the color to indicate whether a projected glucose level for the patient is outside of a target range and/or may not indicate a projected glucose level. The projected glucose level may be received from sensor device 20 or patient device 24 (or another device) may determine the projected glucose level, for example, using a current glucose level received from sensor device 20. For instance, patient device 24 may cause wearable device 22 to display three arrows if the projected glucose level for the patient is greater than the target range. In some instances, patient device 24 may cause wearable device 22 to display the projected glucose level in an orange color if the projected glucose level for the patient is greater than the target range and/or based on a rate of change of the first graphical representation by patient projected glucose level. Patient device 24 may provide more detailed information than the second graphical representation displayed by wearable device 22, which can help provide valuable context to display the current glucose level in an orange color if the projected glucose level for the patient is greater than the target range and/or based on a rate of change of the current glucose level. Configuring patient device 24 and wearable device 22 to display different graphical representations may help to reduce an amount of time patient 12 spends reviewing his or her glucose level, which may reduce a power consumption of patient device 24 and/or wearable device 22. Moreover, reducing an amount of time patient 12 spends reviewing his or her glucose level may improve a satisfaction of patient 12 with system 10A.

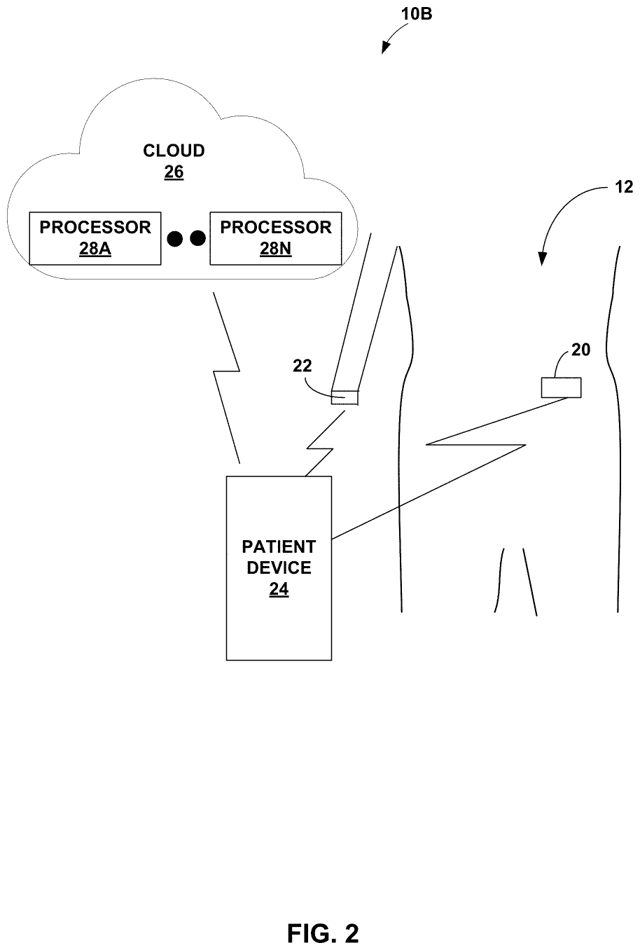

FIG. 2 is a block diagram illustrating another example system for delivering or guiding therapy dosage, in accordance with one or more examples described in this disclosure. FIG. 2 illustrates system 10B that is similar to system 10A of FIG. 1. However, in system 10B, patient 12 may not have insulin pump 14. Rather, patient 12 may utilize a manual injection device (e.g., an insulin pen or a syringe) to deliver insulin. For example, rather than insulin pump 14 automatically delivering insulin, patient 12 (or possible a caretaker of patient 12) may fill a syringe with insulin or set the dosage amount in an insulin pen and inject himself or herself. In this example, patient device 24 may be configured to output an indication of an amount of insulin to the patient for display.

System 10B may be configured to determine a glucose level of patient 12. For example, sensor device 20 may determine a glucose level of patient 12 and output an indication of the glucose level to patient device 24. In this example, patient device 24 may receive the glucose level from sensor device 20, however, in other examples, patient device 24 may determine or estimate the glucose level, receive a patient reported glucose level, or receive an indication of the glucose from another device.

System 10B may determine the first graphical representation to provide addition contextual information that is not displayed in the second graphical representation. For example, the graphical representation may include a graph of glucose levels for patient 12 over time. In some examples, the graph of glucose levels for patient 12 over time may be configured to display different time ranges based on a user input. In contrast, the second graphical representation may provide a current and/or projected glucose level without the additional context information (e.g., the graph). The second graphical representation may be more formed in a way to provide pertinent information in a way that is easier for the user to understand. In this way, a user (e.g., patient 12 or a caretaker of patient 12) may quickly identify pertinent information using the second graphical representation and determine further contextual information using the first graphical representation, which may improve a user satisfaction of system 10B. Moreover, wearable device 22 may comprise a smaller display than patient device 24. As such, system 10B may determine the second graphical display to display less information than the first graphical display to provide information in a format that is readable to a user. Similarly, system 10B may determine the first graphical representation to display more information than the second graphical representation to provide additional context and a richer display of information.

A display of the second graphical representation by wearable device 22 may generally be associated with the user taking immediate action (e.g., provides high level information that is easily consumable by the user). For example, patient device 24 may determine the second graphical representation to include a number of arrows or color to indicate whether a projected glucose level for the patient is outside of a target range. Patient device 24 may determine the second graphical representation to include one or more indicators similar to weather forecasting. For example, the one or more indicators may show a probability of going low in the next 10 mins, 20 mins, 30 mins. In some examples, the one or more indicators may directly show a projectile trace with a confidence interval shown, for example, as cones. The projected glucose level may be received from sensor device 20 or patient device 24 (or another device) may determine the projected glucose level, for example, using a current glucose level received from sensor device 20. For instance, patient device 24 may cause wearable device 22 to display three arrows if the projected glucose level for the patient is greater than the target range. In some instances, patient device 24 may cause wearable device 22 to display the projected glucose level in an orange color if the projected glucose level for the patient is greater than the target range and/or based on a rate of change of the first graphical representation by patient projected glucose level. Patient device 24 may provide more detailed information than the second graphical representation displayed by wearable device 22, which can help provide valuable context to display the projected glucose level in an orange color if the current glucose level for the patient is greater than the target range and/or based on a rate of change of the current glucose level. Configuring patient device 24 and wearable device 22 to display different graphical representations may help to reduce an amount of time patient 12 spends reviewing his or her glucose level, which may reduce a power consumption of patient device 24 and/or wearable device 22. Moreover, reducing an amount of time patient 12 spends reviewing his or her glucose level may improve a satisfaction of patient 12 with system 10B.

FIG. 3 is a block diagram illustrating another example system for delivering or guiding therapy dosage, in accordance with one or more examples described in this disclosure. FIG. 3 illustrates system 10C that is similar to system 10A of FIG. 1 and system 10B of FIG. 2. In system 10C, patient 12 may not have insulin pump 14. Rather, patient 12 may utilize injection device 30 to deliver insulin. For example, rather than insulin pump 14 automatically delivering insulin, patient 12 (or possible a caretaker of patient 12) may utilize injection device 30 to inject himself or herself.

Injection device 30 may be different than a syringe because injection device 30 may be a device that can communicate with patient device 24 and/or other devices in system 10C. Also, injection device 30 may include a reservoir, and based on information indicative of how much therapy dosage to deliver may be able to dose out that much insulin for delivery. For example, injection device 30 may automatically set the amount of insulin based on the information received from patient device 24. In some examples, injection device 30 may be similar to insulin pump 14, but not worn by patient 12. One example of injection device 30 is an insulin pen, sometimes embodied and equipped with electronics to render injection device 30 as a smart insulin pen capable of communicating and/or interacting with other components such as the patient device 24 or system 10C. Another example of injection device 30 may be an insulin pen with a smart cap, where the smart cap can be used to set particular doses of insulin and/or communicate with other components such as the patient device 24 or system 10C.

System 10C may be configured to determine a glucose level of patient 12. For example, sensor device 20 may determine a glucose level of patient 12 and output an indication of the glucose level to patient device 24. In this example, patient device 24 may receive the glucose level from sensor device 20, however, in other examples, patient device 24 may determine or estimate the glucose level, receive a patient reported glucose level, or receive an indication of the glucose from another device.

System 10C may determine a first graphical representation of the glucose level for display on patient device 24 based on the glucose level of patient 12. For example, patient device 24 may display a graph of glucose levels for patient 12 over time. System 10C may determine a second graphical representation of the glucose level for display on wearable device 22 based on a target range of glucose levels and output an instruction to cause wearable device 22 to display the second graphical representation. The second graphical representation may be different from the first graphical representation. For example, patient device 24 may determine the second graphical representation to include a number of arrows or color to indicate whether a projected glucose level for the patient is outside of a target range. For instance, patient device 24 may cause wearable device 22 to display three arrows if the projected glucose level for the patient is greater than the target range (or greater than the target range by a threshold amount). In some instances, patient device 24 may cause wearable device 22 to display the projected glucose level in an orange color if the projected glucose level for the patient is greater than the target range. In some instances, patient device 24 may cause wearable device 22 to display the current glucose level in an orange color if the projected glucose level for the patient is greater than the target range. Configuring patient device 24 and wearable device 22 to display different graphical representations may help to reduce an amount of time patient 12 spends reviewing his or her glucose level, which may reduce a power consumption of patient device 24 and/or wearable device 22. For instance, patient 12 may more quickly inject himself or herself using injection device 30, which reduce a time to provide therapy. Reducing a time to provide therapy may help to improve a therapy provided by delivering insulin more quickly to patient 12. Moreover, reducing an amount of time patient 12 spends reviewing his or her glucose level may improve a satisfaction of patient 12 with system 10C.

The above examples described insulin pump 14, a syringe, and injection device 30 as example ways in which to deliver insulin. In this disclosure, the term “insulin delivery device” may generally refer to any device used to deliver insulin. Examples of insulin delivery device include insulin pump 14, a syringe, and injection device 30. As described, the syringe may be a device used to inject insulin but is not necessarily capable of communicating or dosing a particular amount of insulin. Injection device 30, however, may be a device used to inject insulin that may be capable of communicating with other devices (e.g., via Wi-Fi™, IEEE 802.11, Bluetooth™, or BLE) or may be capable of dosing a particular amount of insulin. Injection device 30 may be powered (e.g., battery powered) device, and the syringe may be a device that requires no electrical power.

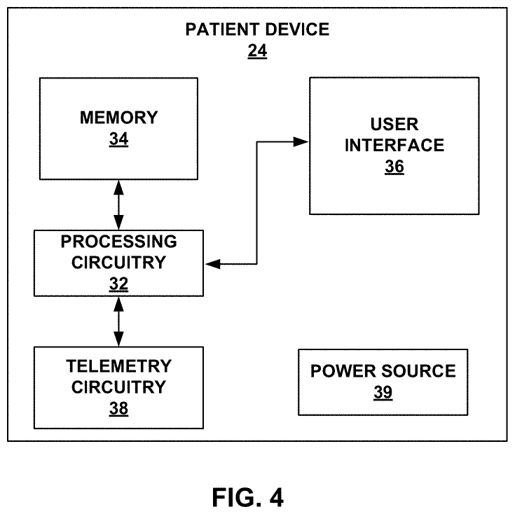

FIG. 4 is a block diagram illustrating an example of a patient device, in accordance with one or more examples described in this disclosure. While patient device 24 may generally be described as a hand-held computing device, patient device 24 may be a notebook computer, a cell phone, or a workstation, for example. In some examples, patient device 24 may be a mobile device, such as a smartphone or a tablet computer. In such examples, patient device 24 may execute an application that allows patient device 24 to perform example techniques described in this disclosure. In some examples, patient device 24 may be specialized controller for communicating with insulin pump 14.

Although the examples are described with one patient device 24, in some examples, patient device 24 may be a combination of different devices (e.g., mobile device and a controller). For instance, the mobile device may provide access to processing circuitry 28 of cloud 26 through Wi-Fi™ or carrier network and the controller may provide access to insulin pump 14. In such examples, the mobile device and the controller may communicate with one another through, for example, Wi-Fi™, Bluetooth™, and/or BLE. Various combinations of a mobile device and a controller together forming patient device 24 are possible and the example techniques should not be considered limited to any one particular configuration.

As illustrated in FIG. 4, patient device 24 may include a processing circuitry 32, memory 34, user interface 36, telemetry circuitry 38, and power source 39. Memory 34 may store program instructions that, when executed by processing circuitry 32, cause processing circuitry 32 to provide the functionality ascribed to patient device 24 throughout this disclosure.

In some examples, memory 34 of patient device 24 may store a plurality of parameters, such as amounts of insulin to deliver, target glucose level, time of delivery etc. Processing circuitry 32 (e.g., through telemetry circuitry 38) may output the parameters stored in memory 34 to insulin pump 14 or injection device 30 for delivery of insulin to patient 12. In some examples, processing circuitry 32 may execute a notification application, stored in memory 34, that outputs notifications to patient 12, such as notification to take insulin, amount of insulin, and time to take the insulin, via user interface 36.

Memory 34 may include any volatile, non-volatile, fixed, removable, magnetic, optical, or electrical media, such as RAM, ROM, hard disk, removable magnetic disk, memory cards or sticks, NVRAM, EEPROM, flash memory, and the like. Processing circuitry 32 can take the form one or more microprocessors, DSPs, ASICs, FPGAs, programmable logic circuitry, or the like, and the functions attributed to processing circuitry 32 herein may be embodied as hardware, firmware, software or any combination thereof.

User interface 36 may include a button or keypad, lights, a speaker for voice commands, and a display, such as a liquid crystal (LCD). In some examples the display may be a touchscreen. As discussed in this disclosure, processing circuitry 32 may present and receive information relating to therapy via user interface 36. For example, processing circuitry 32 may receive patient input via user interface 36. The patient input may be entered, for example, by pressing a button on a keypad, entering text, or selecting an icon from a touchscreen. The patient input may be information indicative of food that patient 12 eats, such as for the initial learning phase, whether patient 12 took the insulin (e.g., through the syringe or injection device 30), and other such information.

Telemetry circuitry 38 includes any suitable hardware, firmware, software or any combination thereof for communicating with another device, such as cloud 26, insulin pump 16 or injection device 30, as appliable, wearable device 22, and sensor device 20. Telemetry circuitry 38 may receive communication with the aid of an antenna, which may be internal and/or external to patient device 24. Telemetry circuitry 38 may be configured to communicate with another computing device via wireless communication techniques, or direct communication through a wired connection. Examples of local wireless communication techniques that may be employed to facilitate communication between patient device 24 and another computing device include RF communication according to, for example, Wi-Fi™, IEEE 802.11, Bluetooth™, or BLE specification sets, infrared communication, e.g., according to an IrDA standard, or other standard or proprietary telemetry protocols. Telemetry circuitry 38 may also provide connection with carrier network for access to cloud 26. In this manner, other devices may be capable of communicating with patient device 24.

Power source 39 delivers operating power to the components of patient device 24. In some examples, power source 39 may include a battery, such as a rechargeable or non-rechargeable battery. A non-rechargeable battery may be selected to last for several years, while a rechargeable battery may be inductively charged from an external device, e.g., on a daily or weekly basis. Recharging of a rechargeable battery may be accomplished by using an alternating current (AC) outlet or through proximal inductive interaction between an external charger and an inductive charging coil within patient device 24.

Processing circuitry 32 may be configured to determine a glucose level of patient 12. For example, telemetry circuitry 38 may receive the glucose level from sensor device 20. In some examples, processing circuitry 32 may determine or estimate the glucose level, telemetry user interface 36 may receive a patient reported glucose level, or telemetry circuitry 38 may receive an indication of the glucose from another device. Sensor device 20 may include one or more sensing elements configured to be inserted at least partially inside patient 12.

Processing circuitry 32 may determine a first graphical representation of the glucose level for display on user interface 36 based on the glucose level of patient 12. For example, processing circuitry 32 may cause user interface 36 to display a graph of glucose levels for patient 12 over time that includes the current glucose level and one or more previous glucose levels of patient 12. The second graphical representation may not include the graph. Processing circuitry 32 may determine a second graphical representation of the glucose level for display on wearable device 22 based on a target range of glucose levels and output an instruction to cause wearable device 22 to display the second graphical representation. The second graphical representation may be different from the first graphical representation. For example, processing circuitry 32 may determine the second graphical representation to include a projected glucose level without one or more previous glucose levels of patient 12. In another example, processing circuitry 32 may determine the second graphical representation to include the current glucose level with an indication of whether a projected glucose level is rising or falling (e.g., arrows separate from the current glucose level and/or the current glucose level in a particular color).

For example, processing circuitry 32 may determine the second graphical representation to include a number of arrows or color to indicate whether a projected glucose level for the patient is outside of a target range. For example, processing circuitry 32 may determine a graphical feature to include in the second graphical representation based on whether a projected glucose level for the patient is less than the target range, within the target range, or greater than the target range. Processing circuitry 32 may determine the projected glucose level as a current glucose level, for instance, a glucose level received from sensor device 20. In some examples, processing circuitry 32 may determine the projected glucose level based on based on the glucose level and based further on one or more previous glucose levels for the patient.

For example, processing circuitry 32 may determine a number of arrows to include in the second graphical representation based on whether the projected glucose level is less than the target range, within the target range, or greater than the target range. For instance, processing circuitry 32 may cause wearable device 22 to display three vertical arrows if the projected glucose level for patient 12 is greater than the target range. In some examples, one or more arrows may pointing straight up (e.g., vertical) or point at an angle (e.g., 45 degrees to 90 degrees).

The one or more arrows may comprise one, two, or more than three arrows that is based on a rate of change of the projected glucose level for patient 12. For example, processing circuitry 32 may cause wearable device 22 to display one arrow pointing upward (e.g., vertical or at an angle) if the projected glucose level for patient 12 is greater than the target range and the projected glucose level for patient 12 has a rate of change (e.g., a positive rate of change) with a magnitude that is within a first range of threshold values. In this example, processing circuitry 32 may cause wearable device 22 to display two arrows pointing upward (e.g., vertical and/or at an angle) if the projected glucose level for patient 12 is greater than the target range and the projected glucose level for patient 12 has a rate of change with a magnitude that is within a second range of threshold values that is greater than the first range of threshold values (e.g., the rate of change for two arrows goes up faster than the rate of change for one arrow). Processing circuitry 32 may cause wearable device 22 to display three arrows pointing upward (e.g., vertical and/or at an angle) if the projected glucose level for patient 12 is greater than the target range and the projected glucose level for patient 12 has a rate of change with a magnitude that is within a third range of threshold values, where the third range of threshold values is greater than the second range of threshold values. In this example, processing circuitry 32 may cause wearable device 22 to display four arrows pointing upward (e.g., vertical and/or at an angle) if the projected glucose level for patient 12 is greater than the target range and the projected glucose level for patient 12 has a rate of change with a magnitude that is within a fourth range of threshold values, where the fourth range of threshold values is greater than the third range of threshold values.

Processing circuitry 32 may cause wearable device 22 to display one or more vertical arrows pointing straight down (e.g., vertical) or point at an angle (e.g., 45 degrees to 90 degrees) if the projected glucose level for patient 12 is less than the target range. For example, processing circuitry 32 may cause wearable device 22 to display one arrow pointing downward (e.g., vertical or at an angle) if the projected glucose level for patient 12 is less than the target range and the projected glucose level for patient 12 has a rate of change (e.g., a negative rate of change) with a magnitude that is less than a first range of threshold values. In this example, processing circuitry 32 may cause wearable device 22 to display two arrows pointing downward (e.g., vertical and/or at an angle) if the projected glucose level for patient 12 is less than the target range and the projected glucose level for patient 12 has a rate of change with a magnitude that is within a second range of threshold values that is greater than the first range of threshold values (e.g., the rate of change for two arrows goes down faster than the rate of change for one arrow).

Processing circuitry 32 may determine a color to display a projected glucose level in the second graphical representation based on whether the projected glucose level is less than the target range, within the target range, or greater than the target range. The first graphical representation may not include the number of arrows or the color to indicate whether a projected glucose level for the patient is outside of a target range and/or may not indicate a projected glucose level. For instance, processing circuitry 32 may cause wearable device 22 to display the projected glucose level in an orange color if the projected glucose level for patient 12 is greater than the target range. Processing circuitry 32 may cause wearable device 22 to display the projected glucose level in a white color if the projected glucose level for patient 12 is within the target range. In some examples, processing circuitry 32 may cause wearable device 22 to display the projected glucose level in a red color if the projected glucose level for patient 12 is less than the target range.

Processing circuitry 32 may determine a color to display a projected glucose level in the second graphical representation based on a rate of change of the projected glucose level for patient 12. For example, processing circuitry 32 may cause wearable device 22 to display a projected glucose level in a first color (e.g., white) if the projected glucose level for patient 12 has a rate of change (e.g., a positive or negative rate of change) with a magnitude that is within than a first range of threshold values. In this example, processing circuitry 32 may cause wearable device 22 to display a projected glucose level in a second color (e.g., orange) if the projected glucose level for patient 12 has a rate of change with a magnitude that is within a second range of threshold values that is greater than the first range of threshold values. Processing circuitry 32 may cause wearable device 22 to display a projected glucose level in a third color (e.g., red) if the projected glucose level for patient 12 has a rate of change with a magnitude that is within a third range of threshold values, where the third range of threshold values is greater than the second range of threshold values.

Processing circuitry 32 may cause wearable device 22 to display one or more vertical arrows pointing straight down (e.g., vertical) or point at an angle (e.g., 45 degrees to 90 degrees) if the projected glucose level for patient 12 is less than the target range. For example, processing circuitry 32 may cause wearable device 22 to display one arrow pointing downward (e.g., vertical or at an angle) if the projected glucose level for patient 12 is less than the target range and the projected glucose level for patient 12 has a rate of change (e.g., a negative rate of change) with a magnitude that is less than a first threshold value. In this example, processing circuitry 32 may cause wearable device 22 to display two arrows pointing downward (e.g., vertical and/or at an angle) if the projected glucose level for patient 12 is less than the target range and the projected glucose level for patient 12 has a rate of change with a magnitude that is greater than the first threshold value (e.g., the rate of change for two arrows goes down faster than the rate of change for one arrow).

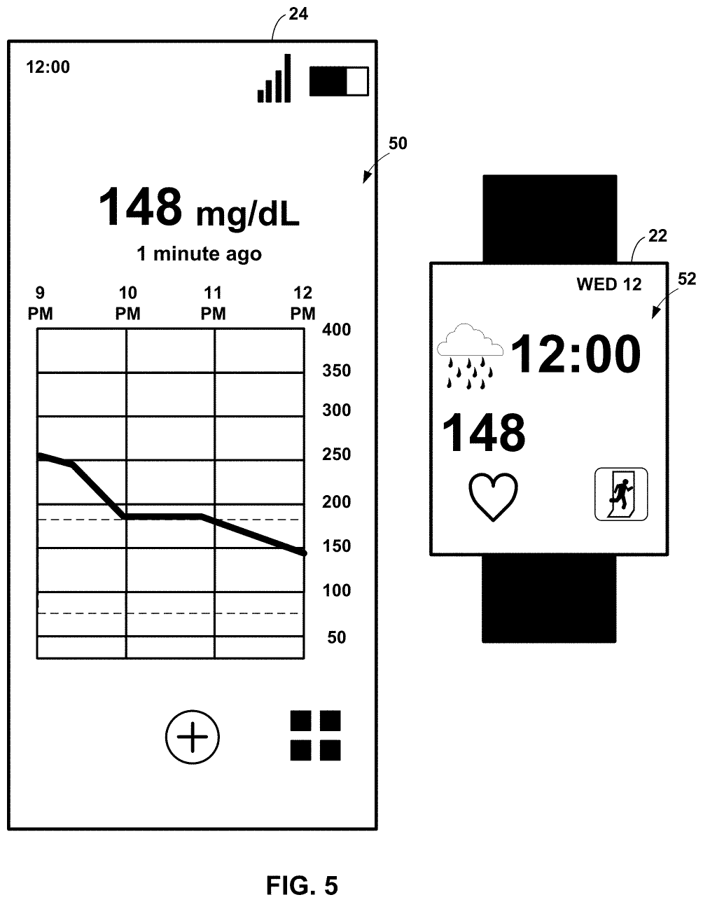

FIG. 5 is a conceptual diagram illustrating a first example of a patient device 24 and a wearable device 22 displaying different graphical representations, in accordance with one or more examples described in this disclosure. FIG. 5 is discussed with reference to FIGS. 1-4 for example purposes only. In the example of FIG. 5, patient device 24 displays a first graphical representation 50 that includes a graph of glucose levels over time that includes the glucose level and one or more previous glucose levels of patient 12. In this example, patient device 24 is configured to cause wearable device 22 display a second graphical representation 52. As shown, first graphical representation 50 and second graphical representation 52 are different. For instance, second graphical representation 52 does not include previous glucose levels of patient 12 (e.g., the graph of first graphical representation 50).

Processing circuitry 32 may determine a color (e.g., white, orange, or red) to display the projected glucose level (e.g., 148 mg/dL) in second graphical representation 52 based on whether the projected glucose level is less than the target range, within the target range, or greater than the target range. Processing circuitry 32 may determine the color to display the projected glucose level (e.g., 148 mg/dL) in second graphical representation 52 based on a rate of change of the projected glucose level for patient 12. Processing circuitry 32 may determine the color to display the projected glucose level (e.g., 148 mg/dL) in second graphical representation 52 based on a combination of a rate of change of the projected glucose level for patient 12 and whether the projected glucose level is less than the target range, within the target range, or greater than the target range.

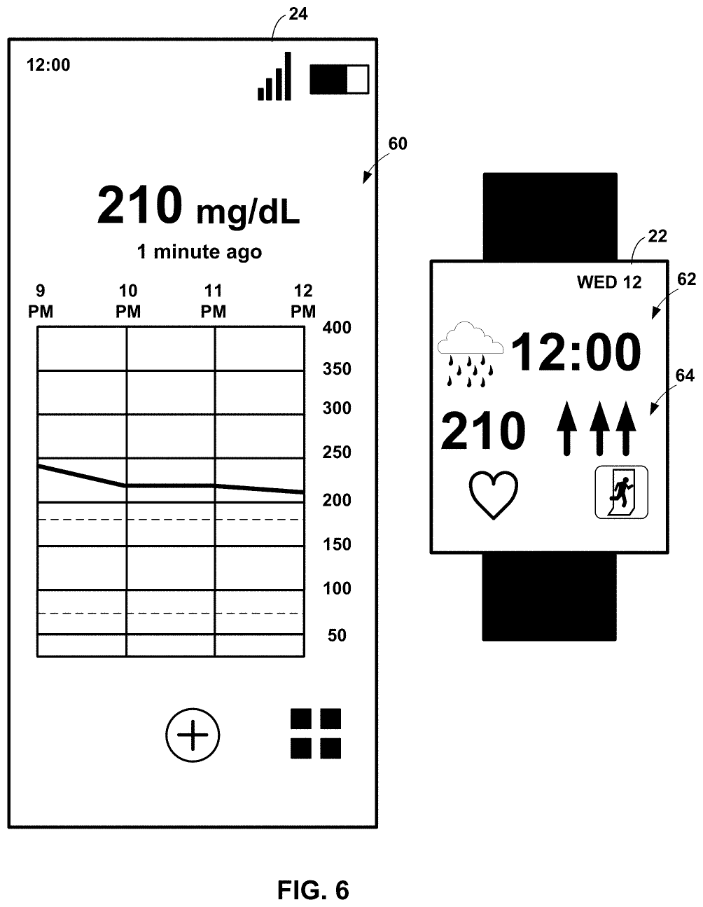

FIG. 6 is a conceptual diagram illustrating a second example of a patient device 24 and a wearable device 22 displaying different graphical indications, in accordance with one or more examples described in this disclosure. FIG. 6 is discussed with reference to FIGS. 1-5 for example purposes only. In the example of FIG. 6, patient device 24 displays a first graphical representation 60 that includes a graph of glucose levels over time that includes the glucose level and one or more previous glucose levels of patient 12. In this example, patient device 24 is configured to cause wearable device 22 display a second graphical representation 62. As shown, first graphical representation 60 and second graphical representation 62 are different. For instance, second graphical representation 62 does not include previous glucose levels of patient 12 (e.g., the graph of first graphical representation 60).

In the example of FIG. 6, second graphical representation 62 includes a number of arrows associated with whether the projected glucose level is less than the target range, within the target range, or greater than the target range. For example, patient device 24 may determine the number of arrows to include in second graphical representation 62 based on whether the projected glucose level for patient 12 is less than the target range, within the target range, or greater than the target range. For instance, patient device 24 may determine to include three arrows 64 in second graphical representation 62 when the projected glucose level for patient 12 is greater than the target range. In some examples, the number of arrows presented may represent the projected rate of change of the projected glucose value for patient 12. In some examples, the angle of arrows 64 may represent the projected rate of change for the projected glucose value for patient 12 (e.g., arrows at 90 degrees representing a higher rate of change than arrows at 45 degrees). Processing circuitry 32 may determine the number of arrows to display in second graphical representation 62 based on a rate of change of the projected glucose level for patient 12. In some examples, processing circuitry 32 may determine the number of arrows in second graphical representation 62 based on a combination of a rate of change of the projected glucose level for patient 12 and whether the projected glucose level is less than the target range, within the target range, or greater than the target range.

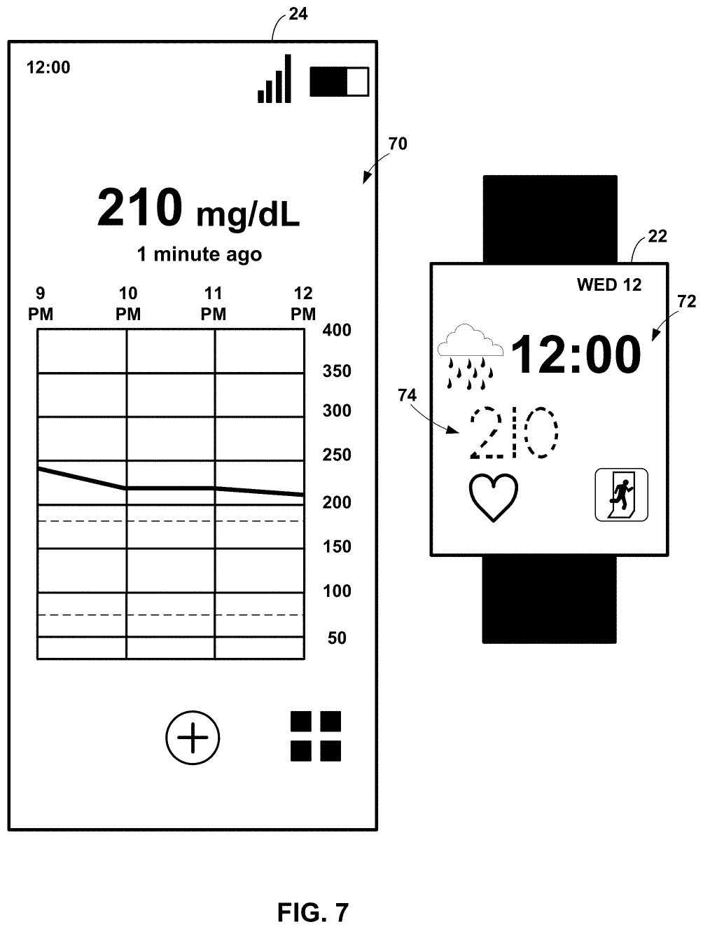

FIG. 7 is a conceptual diagram illustrating a third example of a patient device 24 and a wearable device 22 displaying different graphical indications, in accordance with one or more examples described in this disclosure. FIG. 7 is discussed with reference to FIGS. 1-6 for example purposes only. In the example of FIG. 7, patient device 24 displays a first graphical representation 70 that includes a graph of glucose levels over time that includes the glucose level and one or more previous glucose levels of patient 12. In this example, patient device 24 is configured to cause wearable device 22 display a second graphical representation 72. As shown, first graphical representation 70 and second graphical representation 72 are different. For instance, second graphical representation 72 does not include previous glucose levels of patient 12 (e.g., the graph of first graphical representation 70).

In the example of FIG. 7, second graphical representation 72 includes a projected glucose level indicator 74 for patient 12 that is displayed in a color associated with whether the projected glucose level is less than the target range, within the target range, or greater than the target range. For example, patient device 24 may determine a color for projected glucose level indicator 74 based on whether the projected glucose level for patient 12 is less than the target range, within the target range, or greater than the target range. For instance, patient device 24 may determine the color of projected glucose level indicator 74 to be orange when the projected glucose level for patient 12 is greater than the target range. Patient device 24 may determine the color of projected glucose level indicator 74 to be white when the projected glucose level for patient 12 is within the target range. In some examples, patient device 24 may determine the color of projected glucose level indicator 74 to be red when the projected glucose level for patient 12 is less than the target range. As shown in FIG. 7, second graphical representation 72 may include the current sensor glucose value (e.g., 210 mg/dL) displayed in a color representing a projected glucose level. In other examples, second graphical representation 72 may include the projected sensor glucose value.

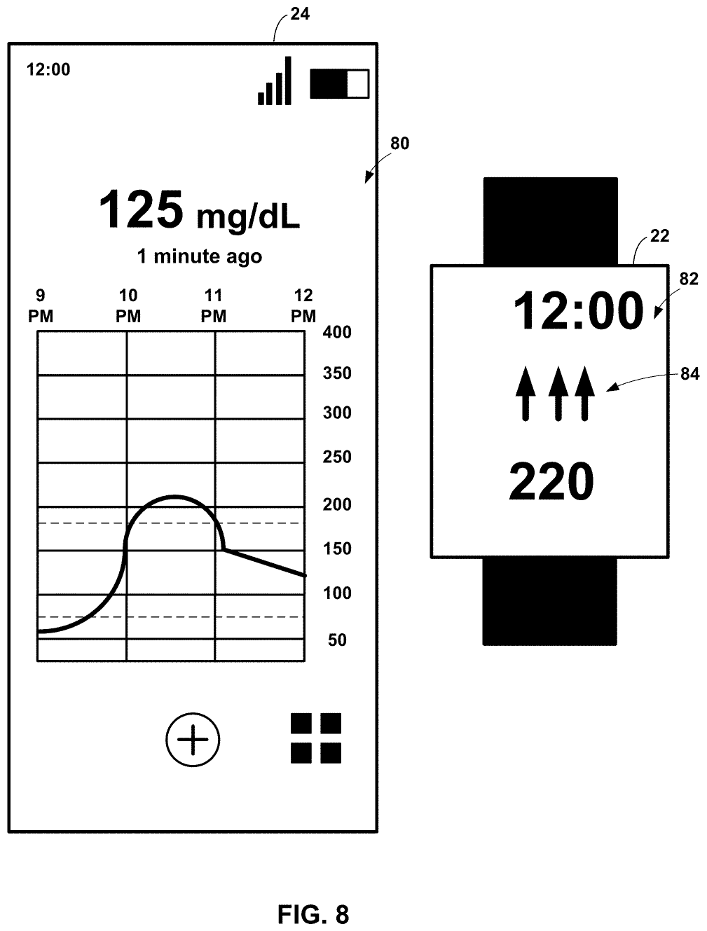

FIG. 8 is a diagram illustrating example graphical user interfaces of two patient devices (e.g., patient device 24 and wearable device 22) for displaying real-time (or near real-time) glucose level information, in accordance with one or more examples described in this disclosure. In this example, an application running on patient device 24 (e.g., a smartphone) may generate a first graphical representation 80 (e.g., a first graphical user interface) including an indication of the current glucose levels of patient 12 and a graph of the patient's glucose levels over time. FIG. 8 also shows an application running on wearable device 22 (e.g., a smart watch) showing a second graphical representation 84 (e.g., a second graphical user interface), different than first graphical representation 80, with an indication 84 showing how the user glucose levels are trending and/or a projected glucose levels. In this example, FIG. 8 shows the glucose levels quickly trending up (e.g., spiking) with three vertical arrows and a projected glucose level of 220 mg/dL. As shown, the projected glucose level (e.g., 220 mg/dL) and the current glucose level (e.g., 125 mg/dL) are different.

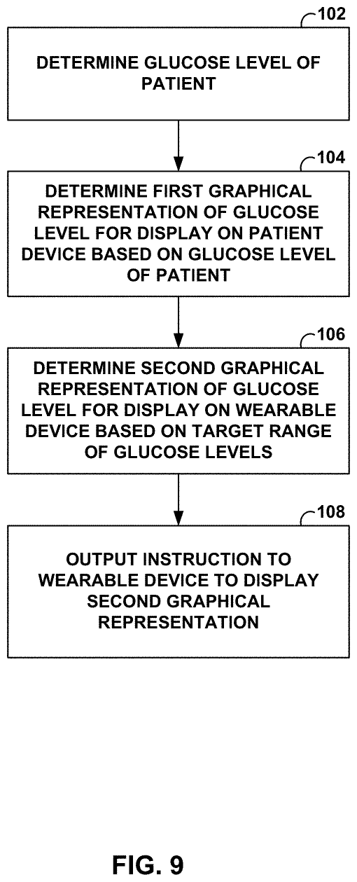

FIG. 9 is a flow chart illustrating an example process, in accordance with one or more examples described in this disclosure. FIG. 9 is discussed with reference to FIGS. 1-8 for example purposes only. The example of FIG. 9 is described with respect to processing circuitry 32, but the example techniques may be performed separately or in combination with processing circuitry 32 by various other components as well.

Processing circuitry 32 may be configured to determine a glucose level of patient 12 (102). For example, telemetry circuitry 38 may receive the glucose level from sensor device 20. In some examples, processing circuitry 32 may determine or estimate the glucose level, telemetry user interface 36 may receive a patient reported glucose level, or telemetry circuitry 38 may receive an indication of the glucose from another device. Sensor device 20 may include one or more sensing elements configured to be inserted at least partially inside patient 12.

Processing circuitry 32 may determine a first graphical representation of the glucose level for display on user interface 36 based on the glucose level of patient 12 (104). For example, processing circuitry 32 may cause user interface 36 to display a graph of glucose levels for patient 12 over time that includes the glucose level and one or more previous glucose levels of patient 12. Processing circuitry 32 may determine a second graphical representation of the glucose level for display on wearable device 22 based on a target range of glucose levels (106) and output an instruction to cause wearable device 22 to display the second graphical representation (108). The second graphical representation may be different from the first graphical representation. For example, processing circuitry 32 may determine the second graphical representation to include a projected glucose level without one or more previous glucose levels of patient 12.

For example, processing circuitry 32 may determine the second graphical representation to include a number of arrows or color to indicate whether a projected glucose level for the patient is outside of a target range. For example, processing circuitry 32 may determine a graphical feature to include in the second graphical representation based on whether a projected glucose level for the patient is less than the target range, within the target range, or greater than the target range. Processing circuitry 32 may determine the projected glucose level as a current glucose level, for instance, a glucose level received from sensor device 20. In some examples, processing circuitry 32 may determine the projected glucose level based on based on the glucose level and based further on one or more previous glucose levels for the patient.

For example, processing circuitry 32 may determine a number of arrows to include in the second graphical representation based on whether the projected glucose level is less than the target range, within the target range, or greater than the target range. For instance, processing circuitry 32 may cause wearable device 22 to display three arrows if the projected glucose level for patient 12 is greater than the target range.

Processing circuitry 32 may determine a color to display a current or projected glucose level in the second graphical representation based on whether the projected glucose level is less than the target range, within the target range, or greater than the target range. For instance, processing circuitry 32 may cause wearable device 22 to display the current or projected glucose level in an orange color if the projected glucose level for patient 12 is greater than the target range. Processing circuitry 32 may cause wearable device 22 to display the current or projected glucose level in a white color if the projected glucose level for patient 12 is within the target range. In some examples, processing circuitry 32 may cause wearable device 22 to display the current or projected glucose level in a red color if the projected glucose level for patient 12 is less than the target range.

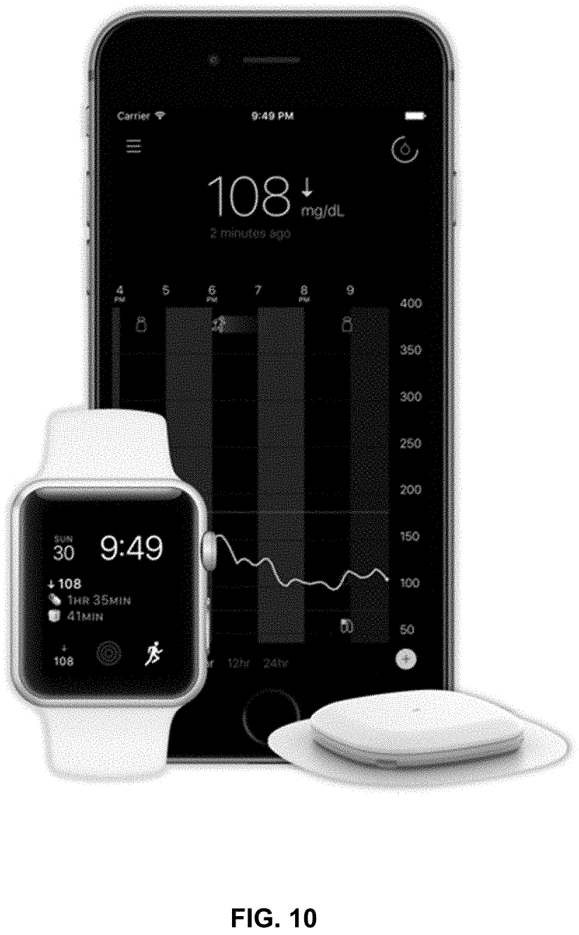

FIG. 10 is a diagram illustrating an example system for monitoring and managing glucose levels of a user, in accordance with one or more examples described in this disclosure. In this example, the system includes a glucose sensor, a first user device (e.g., a smart phone) and corresponding first application, and a second user device (e.g., a smart watch) and corresponding second application. In some examples, the second application can be an extension of the first application (e.g., can display the same or similar information, can receive and/or request input for the first application, can output information for the first application). The glucose sensor and the first and second user devices can be communicatively coupled (e.g., can communicate data amongst the devices). In some examples, the second user device and corresponding second application can comprise a gesture detection system (e.g., can detect arm/hand gestures of the user).

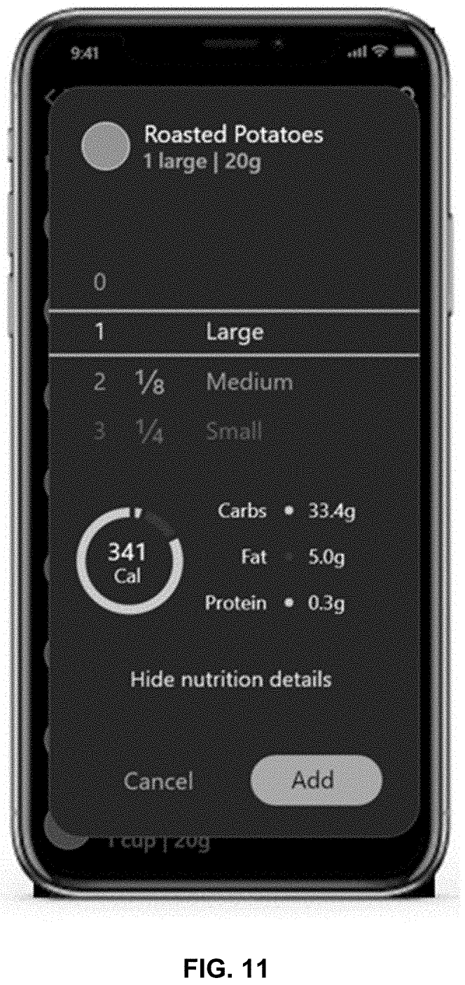

FIG. 11 is a diagram illustrating an example graphical user interface of a patient device for logging meal nutrition information, in accordance with one or more examples described in this disclosure. The graphical user interface shown in FIG. 11 can simplify the logging of macro nutrient information by enabling the user to select the size (e.g., small, medium, or large) of one or more food items (e.g. roasted potatoes) and obtaining, from a database, macro nutrient information about the one or more food items based on the selected size. In some examples, the graphical user interface shown in FIG. 11 is automatically generated when a meal event is detected by a gesture detection system (e.g., the smart watch and corresponding application shown in FIG. 10).

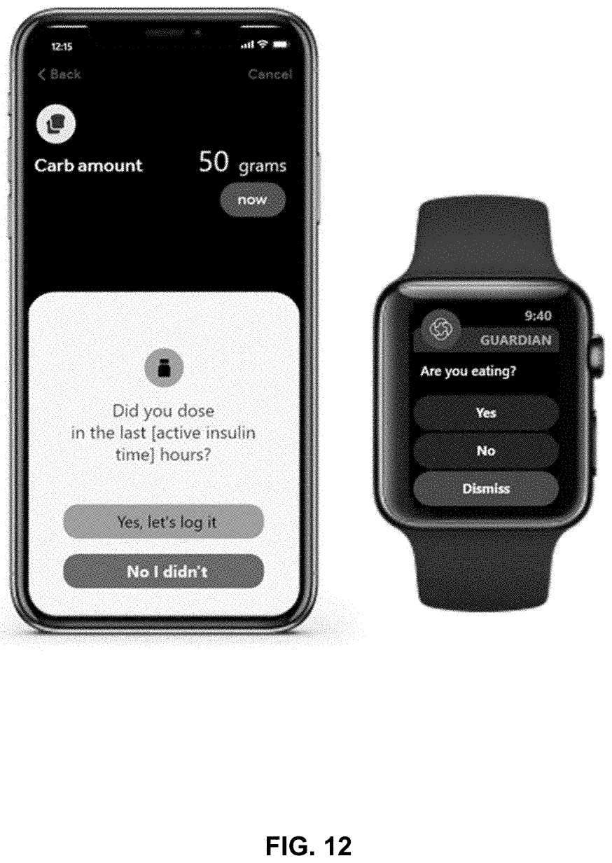

FIG. 12 is a diagram illustrating example user interfaces of two patient devices, in accordance with one or more examples described in this disclosure. In this example, a gesture detection system comprising a first user device (e.g., a smart watch) and a first application running on the first user device. The system detects eating gestures and automatically generates a first alert for confirming whether the user is eating at the first device. If the user indicates that he or she is eating at the first user device, a second user device may generate a second alert for confirming whether the user injected an insulin dose corresponding to the confirmed meal event. In some examples, the first and/or second user device allows the user to enter macro-nutrient information including carbohydrate amount (e.g., as described above with reference to FIG. 11).

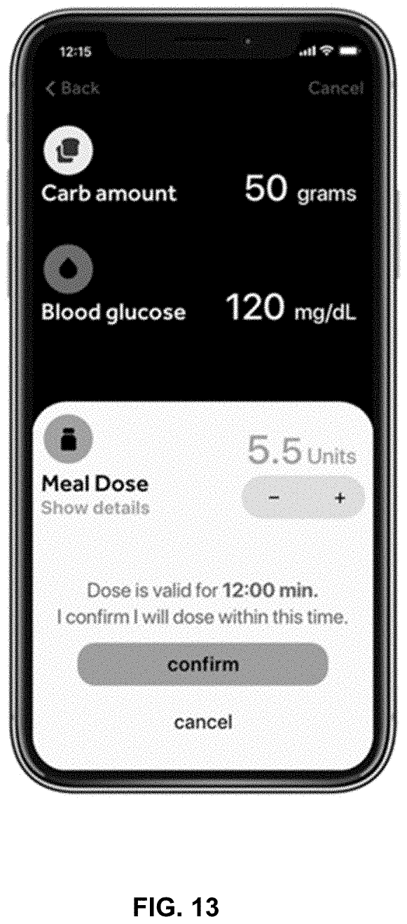

FIG. 13 is a diagram illustrating an example graphical user interface of a patient device including an insulin dose recommendation, in accordance with one or more examples described in this disclosure. The insulin dose recommendation may be generated by the user device based on macronutrient information (e.g., carbohydrate amount) for a meal and the user's current and/or projected glucose levels. In some examples, the graphical user interface shown in FIG. 12 can be generated by the user device in response to the user indicating that he or she did not inject an insulin dose (e.g., as described above with reference to FIG. 12). In some examples, the recommended insulin dose may include an expiration window in which the dose must be administered by the user. If the user does not administer the insulin dose or indicate, via the user interface shown in FIG. 13, that he or she will administer the insulin dose within that expiration window, the user device may generate a new insulin dose recommendation.

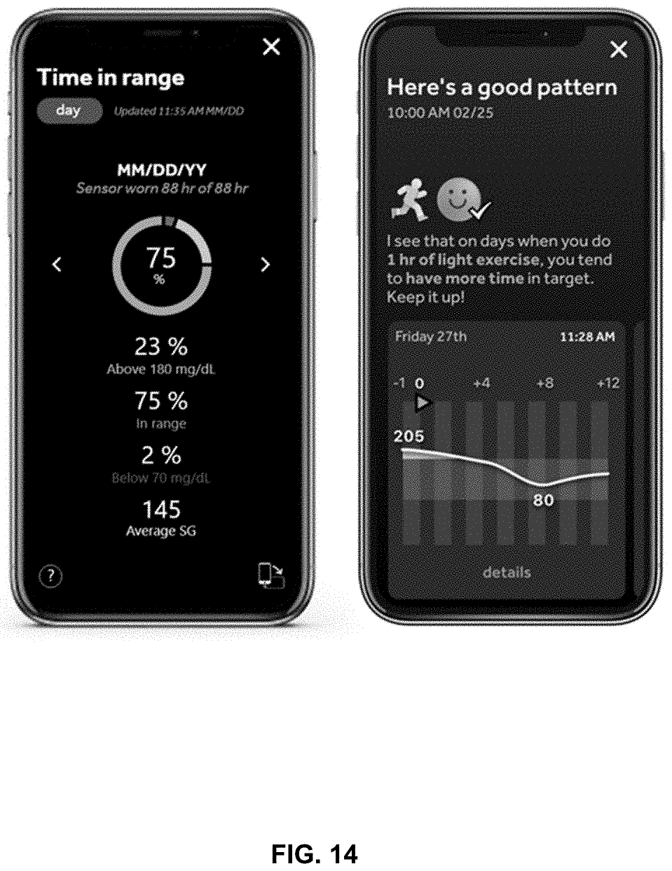

FIG. 14 is a diagram illustrating example user interfaces of a patient device, in accordance with one or more examples described in this disclosure. In this example, the user device can also generate pattern insights on how the user's activities positively or adversely impact glucose levels. The system can also output information about the time or percent of time the user's glucose are in range (e.g., between 180 mg/dL and 70 mg/dL), the time or percent of time the user's glucose are above a high threshold (e.g., 180 mg/dL), the time or percent of time the user's glucose are below a low threshold (e.g., 70 mg/dL), and/or the average glucose level.

The following examples are a non-limiting list of examples in accordance with one or more techniques of this disclosure.

Clause 1: A system for monitoring a patient, the system comprising: a memory; and processing circuitry coupled to the memory and configured to: determine a glucose level of the patient; determine a first graphical representation of the glucose level for display on a patient device based on the glucose level of the patient; determine a second graphical representation of the glucose level for display on a wearable device based on a target range of glucose levels, wherein the second graphical representation is different from the first graphical representation; and output an instruction to cause the wearable device to display the second graphical representation.

Clause 2: The system of clause 1, wherein, to determine the second graphical representation, the processing circuitry is configured to determine a graphical feature to include in the second graphical representation based on whether a projected glucose level for the patient is less than the target range, within the target range, or greater than the target range.

Clause 3: The system of clause 2, wherein the processing circuitry is configured to determine the projected glucose level based on the glucose level and based further on one or more previous glucose levels for the patient.

Clause 4: The system of clauses 1-3, wherein, to determine the second graphical representation, the processing circuitry is configured to determine a number of arrows to include in the second graphical representation based on whether the projected glucose level is less than the target range, within the target range, or greater than the target range.

Clause 5: The system of clauses 1-3, wherein, to determine the second graphical representation, the processing circuitry is configured to determine a color to display the projected glucose level in the second graphical representation based on whether the projected glucose level is less than the target range, within the target range, or greater than the target range.

Clause 6: The system of clauses 1-5, wherein, to determine the first graphical representation of the glucose level, the processing circuitry is configured to determine a graph of glucose levels over time that includes the glucose level and one or more previous glucose levels of the patient.

Clause 7: The system of clause 6, wherein, to determine the second graphical representation, the processing circuitry is configured to determine the second graphical representation to indicate a projected glucose level without the one or more previous glucose levels.

Clause 8: The system of clauses 1-7, further comprising the sensor device, wherein, to determine the glucose level, the processing circuitry is configured to receive an indication of the glucose level from a sensor device.

Clause 9: The system of clause 8, wherein the sensor device comprises one or more sensing elements configured to be inserted at least partially inside the patient.

Clause 10: The system of clauses 1-9, wherein the patient device is configured to display the first graphical representation.

Clause 11: The system of clause 10, wherein, to output the instruction to cause the wearable device to display the second graphical representation, the processing circuitry is configured to output the instructions to cause the wearable device to display the second graphical representation when the patient device displays the first graphical representation.

Clause 12: The system of clauses 1-11, wherein the patient device comprises a mobile phone.

Clause 13: The system of clauses 1-12, wherein the processing circuitry is arranged in the patient device.

Clause 14: The system of clauses 1-13, wherein the wearable device comprises a smart watch.

Clause 15: A method comprising: determining, by processing circuitry, a glucose level of the patient; determining, by the processing circuitry, a first graphical representation of the glucose level for display on a patient device based on the glucose level of the patient; determining, by the processing circuitry, a second graphical representation of the glucose level for display on a wearable device based on a target range of glucose levels, wherein the second graphical representation is different from the first graphical representation; and outputting, by the processing circuitry, an instruction to cause the wearable device to display the second graphical representation.

Clause 16: The method of clause 15, wherein determining the second graphical representation comprises determining a graphical feature to include in the second graphical representation based on whether a projected glucose level for the patient is less than the target range, within the target range, or greater than the target range.

Clause 17: The method of clause 16, further comprising determining the projected glucose level based on the glucose level and based further on one or more previous glucose levels for the patient.

Clause 18: The method of clauses 15-17, wherein determining the second graphical representation comprises determining a number of arrows to include in the second graphical representation based on whether the projected glucose level is less than the target range, within the target range, or greater than the target range.

Clause 19: The method of clauses 15-17, wherein determining the second graphical representation comprises determining a color to display the projected glucose level in the second graphical representation based on whether the projected glucose level is less than the target range, within the target range, or greater than the target range.

Clause 20: A computer readable storage medium storing instructions that, when executed, cause processing circuitry to: determine a glucose level of the patient; determine a first graphical representation of the glucose level for display on a patient device based on the glucose level of the patient; determine a second graphical representation of the glucose level for display on a wearable device based on a target range of glucose levels, wherein the second graphical representation is different from the first graphical representation; and output an instruction to cause the wearable device to display the second graphical representation.

Various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, electrical stimulators, or other devices. The term “processor” or “processing circuitry” may generally refer to any of the foregoing logic circuitry, alone or in combination with other logic circuitry, or any other equivalent circuitry.

In one or more examples, the functions described in this disclosure may be implemented in hardware, software, firmware, or any combination thereof If implemented in software, the functions may be stored on, as one or more instructions or code, a computer-readable medium and executed by a hardware-based processing unit. Computer-readable media may include computer-readable storage media forming a tangible, non-transitory medium. Instructions may be executed by one or more processors, such as one or more DSPs, ASICs, FPGAs, general purpose microprocessors, or other equivalent integrated or discrete logic circuitry. Accordingly, the term “processor,” as used herein may refer to one or more of any of the foregoing structure or any other structure suitable for implementation of the techniques described herein.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including processing circuitry 28 of cloud 26, processing circuitry of patient device 24, processing circuitry of wearable device 22, processing circuitry of insulin pump 14, or some combination thereof. The processing circuitry may be one or more integrated circuits (ICs), and/or discrete electrical circuitry, residing in various locations in the example systems described in this disclosure.

The one or more processors or processing circuitry utilized for example techniques described in this disclosure may be implemented as fixed-function circuits, programmable circuits, or a combination thereof. Fixed-function circuits refer to circuits that provide particular functionality, and are preset on the operations that can be performed. Programmable circuits refer to circuits that can be programmed to perform various tasks, and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits. The processors or processing circuitry may include arithmetic logic units (ALUs), elementary function units (EFUs), digital circuits, analog circuits, and/or programmable cores, formed from programmable circuits. In examples where the operations of the processors or processing circuitry are performed using software executed by the programmable circuits, memory accessible by the processors or processing circuitry may store the object code of the software that the processors or processing circuitry receive and execute.

Various aspects of the disclosure have been described. These and other aspects are within the scope of the following claims.

What is claimed is:

1. A system for monitoring a patient, the system comprising:
a memory; and
processing circuitry coupled to the memory and configured to:
determine a glucose level of the patient;
determine a first graphical representation of the glucose level for display on a patient device based on the glucose level of the patient;
determine a second graphical representation of the glucose level for display on a wearable device based on a target range of glucose levels, wherein the second graphical representation is different from the first graphical representation; and
output an instruction to cause the wearable device to display the second graphical representation.

2. The system of claim 1, wherein, to determine the second graphical representation, the processing circuitry is configured to determine a graphical feature to include in the second graphical representation based on whether a projected glucose level for the patient is less than the target range, within the target range, or greater than the target range.

3. The system of claim 2, wherein the processing circuitry is configured to determine the projected glucose level based on the glucose level and based further on one or more previous glucose levels for the patient.

4. The system of claim 1, wherein, to determine the second graphical representation, the processing circuitry is configured to determine a number of arrows to include in the second graphical representation based on whether the projected glucose level is less than the target range, within the target range, or greater than the target range.

5. The system of claim 1, wherein, to determine the second graphical representation, the processing circuitry is configured to determine a color to display the projected glucose level in the second graphical representation based on whether the projected glucose level is less than the target range, within the target range, or greater than the target range.

6. The system of claim 1, wherein, to determine the first graphical representation of the glucose level, the processing circuitry is configured to determine a graph of glucose levels over time that includes the glucose level and one or more previous glucose levels of the patient.

7. The system of claim 6, wherein, to determine the second graphical representation, the processing circuitry is configured to determine the second graphical representation to indicate a projected glucose level without the one or more previous glucose levels.

8. The system of claim 1, further comprising the sensor device, wherein, to determine the glucose level, the processing circuitry is configured to receive an indication of the glucose level from a sensor device.

9. The system of claim 8, wherein the sensor device comprises one or more sensing elements configured to be inserted at least partially inside the patient.

10. The system of claim 1, wherein the patient device is configured to display the first graphical representation.

11. The system of claim 10, wherein, to output the instruction to cause the wearable device to display the second graphical representation, the processing circuitry is configured to output the instructions to cause the wearable device to display the second graphical representation when the patient device displays the first graphical representation.

12. The system of claim 1, wherein the patient device comprises a mobile phone.

13. The system of claim 1, wherein the processing circuitry is arranged in the patient device.

14. The system of claim 1, wherein the wearable device comprises a smart watch.

15. A method comprising:
determining, by processing circuitry, a glucose level of the patient;
determining, by the processing circuitry, a first graphical representation of the glucose level for display on a patient device based on the glucose level of the patient;
determining, by the processing circuitry, a second graphical representation of the glucose level for display on a wearable device based on a target range of glucose levels, wherein the second graphical representation is different from the first graphical representation; and
outputting, by the processing circuitry, an instruction to cause the wearable device to display the second graphical representation.

16. The method of claim 15, wherein determining the second graphical representation comprises determining a graphical feature to include in the second graphical representation based on whether a projected glucose level for the patient is less than the target range, within the target range, or greater than the target range.

17. The method of claim 16, further comprising determining the projected glucose level based on the glucose level and based further on one or more previous glucose levels for the patient.

18. The method of claim 15, wherein determining the second graphical representation comprises determining a number of arrows to include in the second graphical representation based on whether the projected glucose level is less than the target range, within the target range, or greater than the target range.

19. The method of claim 15, wherein determining the second graphical representation comprises determining a color to display the projected glucose level in the second graphical representation based on whether the projected glucose level is less than the target range, within the target range, or greater than the target range.

20. A computer readable storage medium storing instructions that, when executed, cause processing circuitry to:
determine a glucose level of the patient;
determine a first graphical representation of the glucose level for display on a patient device based on the glucose level of the patient;
determine a second graphical representation of the glucose level for display on a wearable device based on a target range of glucose levels, wherein the second graphical representation is different from the first graphical representation; and
output an instruction to cause the wearable device to display the second graphical representation.
